(19) United States
(12) Patent Application Publication
(10) Pub. No.: US 2024/0315649 A1
(43) Pub. Date: Sep. 26, 2024

(54) BIOMETRIC INFORMATION DISPLAY DEVICE, SYSTEM, BIOMETRIC INFORMATION DISPLAY METHOD, AND BIOMETRIC INFORMATION DISPLAY PROGRAM

(71) Applicants: Yuki Miyano, Tokyo (JP); Taishi Watanabe, Tokyo (JP); Shigenori Kawabata, Tokyo (JP)

(72) Inventors: Yuki Miyano, Tokyo (JP); Taishi Watanabe, Tokyo (JP); Shigenori Kawabata, Tokyo (JP)

(73) Assignees: Ricoh Company, Ltd., Tokyo (JP); National University Corporation Tokyo Medical and Dental University, Tokyo (JP)

(21) Appl. No.: 18/607,964

(22) Filed: Mar. 18, 2024

(30) Foreign Application Priority Data
Mar. 22, 2023 (JP) 2023-046033

(51) Int. Cl. A61B 5/00; A61B 5/248; G06V 40/10

(52) U.S. Cl. CPC A61B 5/743; A61B 5/248; G06V 40/10; A61B 2562/0223

(57) ABSTRACT

A biometric information display device includes circuitry, and a memory storing computer-executable instructions that cause the circuitry to display at least one biological signal waveform including a biological signal measured, and display a graphic representation representing a noise level included in the biological signal waveform at a location corresponding to a peak of the biological signal waveform.

### CROSS-REFERENCE TO RELATED APPLICATIONS

The present application is based on and claims priority under 35 U.S.C. § 119 to Japanese Patent Application No. 2023-046033, filed Mar. 22, 2023, the contents of which are incorporated herein by reference in their entirety.

### BACKGROUND OF THE INVENTION

### 1. Field of the Invention

The present disclosure relates to a biometric information display device, a system, a biometric information display method, and a biometric information display program.

### 2. Description of the Related Art

A method in which current waveforms on multiple virtual electrodes are displayed on a display screen with use of current information reconstructed from biomagnetism measurement data (for example, see Patent Document 1) is known. In a case where a waveform of an electrocardiogram signal from which random noise is removed by averaging is displayed, a method in which a noise level of the electrocardiogram signal is displayed, on a display screen, in a numerical value or a graph in a region provided separately from a region where the waveform is displayed is known (for example, see Patent Document 2).

In the case where the noise level is displayed in the region provided separately from the region where the waveform is displayed, however, it is difficult to intuitively ascertain a degree of influence of noise on the waveform of measurement data. In other words, it is difficult to intuitively compare magnitude (for example, amplitude) of the waveform of the measurement data and magnitude of the noise level.

### Related-Art Document

### Patent Document

[Patent Document 1] Japanese Unexamined Patent Application Publication No. 2021-083479
[Patent Document 2] Japanese Patent No. 6055679

### Non-Patent Document

[Non-Patent Document 1] Andrei Irimia et al., Magnetogastrographic detection of gastric electrical response activity in humans, PHYSICS IN MEDICINE AND BIOLOGY, Phys. Med. Biol. 51, 2006, pp. 1347-1360
[Non-Patent Document 2] Yasuhiro Shirai et al., Magnetocardiography Using a Magnetoresistive Sensor Array, Int Heart J, January 2019, pp. 50-54
[Non-Patent Document 3] Tadashi Masuda et al., Magnetic fields produced by single motor units in human skeletal muscles, Clinical Neurophysiology 110, 1999, pp. 384-389

### SUMMARY OF THE INVENTION

According to an embodiment of the present disclosure, there is provided a biometric information display device including circuitry, and a memory storing computer-executable instructions that cause the circuitry to display at least one biological signal waveform including a biological signal measured, and display a graphic representation representing a noise level included in the biological signal waveform at a location corresponding to a peak of the biological signal waveform.

### BRIEF DESCRIPTION OF THE DRAWINGS

FIG. 1 is a block diagram illustrating an example of a biometric information measurement device including a biometric information display device according to a first embodiment of the present disclosure;

FIG. 2 is a diagram that explains an example model of nerve action currents;

FIG. 3 is a diagram that explains an example in which virtual electrodes and bioelectric currents are superimposed over a morphological image of a living body and displayed a superimposed image;

FIG. 4 is a diagram that explains an example of current waveforms and graphic representations representing noise levels displayed on the display device of FIG. 1;

FIG. 5 is a diagram that explains another example of the graphic representation representing the noise level displayed on the display device;

FIG. 6 is a diagram that explains an example of a method of calculating the noise level illustrated in FIG. 4;

FIG. 7 is a flowchart illustrating an example of processing for displaying the current waveform and the graphic representation representing the noise level on the display device by a data processing device of FIG. 1;

FIG. 8 is a block diagram illustrating a hardware configuration example of the data processing device in FIG. 1;

FIG. 9 is a diagram that explains an example of current waveforms and graphic representations representing noise levels displayed on the display device in a second embodiment of the present disclosure;

FIG. 10 is a diagram that explains yet another example of the graphic representation representing the noise level displayed on the display device; and

FIG. 11 is a diagram that explains an example of current waveforms and graphic representations representing noise levels displayed on the display device in a third embodiment of the present disclosure.

### DESCRIPTION OF THE EMBODIMENTS

The disclosed technique has been made in view of the above problem, and an object of the present disclosure is to enable a user to intuitively ascertain a degree of influence of noise on a current waveform of a biological signal.

Hereinafter, embodiments will be described with reference to the drawings. In the drawings, the same components are denoted by the same reference numerals, and a duplicate description thereof may be omitted.

### First Embodiment

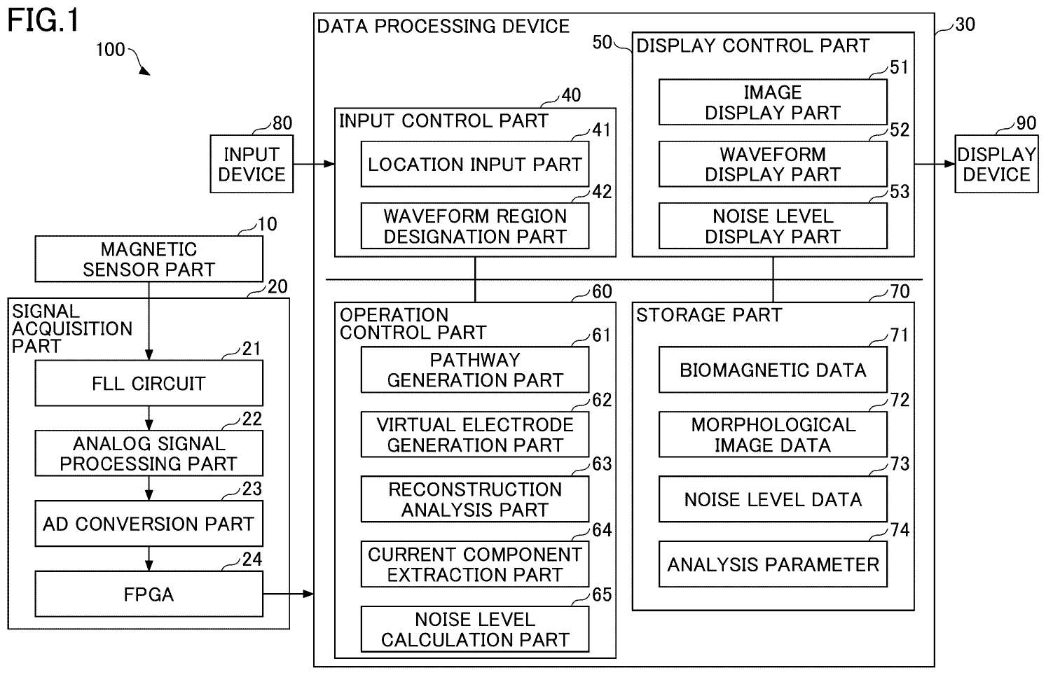

FIG. 1 is a block diagram illustrating an example of a biometric information measurement device including a biometric information display device according to a first embodiment of the present disclosure. For example, a biometric information measurement device 100 illustrated in FIG. 1 includes a magnetic sensor part 10, a signal acquisition part 20, a data processing device 30, an input device 80, and a display device 90.

The magnetic sensor part 10 and the signal acquisition part 20 are example parts of a biomagnetism measurement device. The data processing device 30 is a computer such as a personal computer (PC) or a server, and is an example of a biometric information display device. The biometric information measurement device 100 including the biomagnetism measurement device and the biometric information display device is an example of a system. The display device 90 can display a morphological image of a subject, such as an X-ray image, a current waveform of a biological signal, and the like. The current waveform is an example of a biological signal waveform.

The signal acquisition part 20 includes a flux locked loop (FLL) circuit 21, an analog signal processing part 22, an analog-to-digital (AD) converter 23, and a field-programmable gate array (FPGA) 24. For example, the magnetic sensor part 10 and the signal acquisition part 20 are installed in a magnetically shielded room, and the data processing device 30, the input device 80, and the display device 90 are installed outside the shielded room.

For example, the magnetic sensor part 10 includes superconducting quantum interference device (SQUID) magnetic sensors. Note that the magnetic sensor part 10 may include a magnetic sensor of another type such as a magneto resistive (MR) sensor or an optically pumped atomic magnetometer (OPAM) sensor, instead of the SQUID magnetic sensor.

A biological signal measured by the magnetic sensor part 10 is a signal derived from skeletal muscles, a signal derived from cardiac muscles, a signal derived from smooth muscles, or a signal derived from nerves of a living body. For example, the skeletal muscle is a muscle of a palm, a forearm, an upper arm, a thigh, a lower leg, or the like. For example, the cardiac muscle is a muscle constituting the heart. For example, smooth muscle is a muscle that produces gastric contractions and intestinal peristalsis. For example, an example of measuring a magnetic field of a smooth muscle of a stomach or the like by the SQUID magnetic sensor is described in Non-Patent Document 1. An example of measuring a magnetic field of a myocardium by the SQUID magnetic sensor is described in Non-Patent Document 2. An example of measuring a magnetic field of a skeletal muscle by the SQUID magnetic sensor is described in Non-Patent Document 3.

For example, a signal derived from nerves may be generated from nerves that can be stimulated from a body surface. Each embodiment described below can be applied to current waveforms generated from a biological signal derived from skeletal muscles, cardiac muscles, smooth muscles, or nerves measured by the SQUID magnetic sensor.

The data processing device 30 includes an input control part 40, a display control part 50, an operation control part 60, and a storage part 70. For example, the functions of the display control part 50 are achieved by a biometric information display program executed by a processor such as a central processing unit (CPU) mounted in the data processing device 30 implementing a biometric information display method in cooperation with hardware.

The biometric information measurement device 100 is used as a magnetoencephalograph (MEG), a magnetocardiograph (MCG), a magnetospinograph (MSG), or the like. Note that the biometric information measurement device 100 may be applied to magnetic measurement of nerves or muscles other than the spinal cord.

The magnetic sensor part 10 measures magnetism generated by the subject and outputs the measured magnetism as a voltage. The magnetic sensor part 10 includes, for example, SQUID magnetic sensors which are installed to face a target portion of the subject for magnetic measurement, the subject lying on a bed. The FLL circuit 21 improves a dynamic range of a signal by linearizing each of nonlinear magnetic voltage characteristics measured by the SQUID magnetic sensors.

The analog signal processing part 22 amplifies the linearized analog signal output from the FLL circuit 21 and performs filtering processing and the like on the amplified analog signal. The AD converter 23 converts the filtered analog signal (magnetic signal) into a digital value. The FPGA 24 further performs filtering, thinning, and the like on the magnetic data digitized by the AD converter 23 to transfer the magnetic data to the data processing device 30.

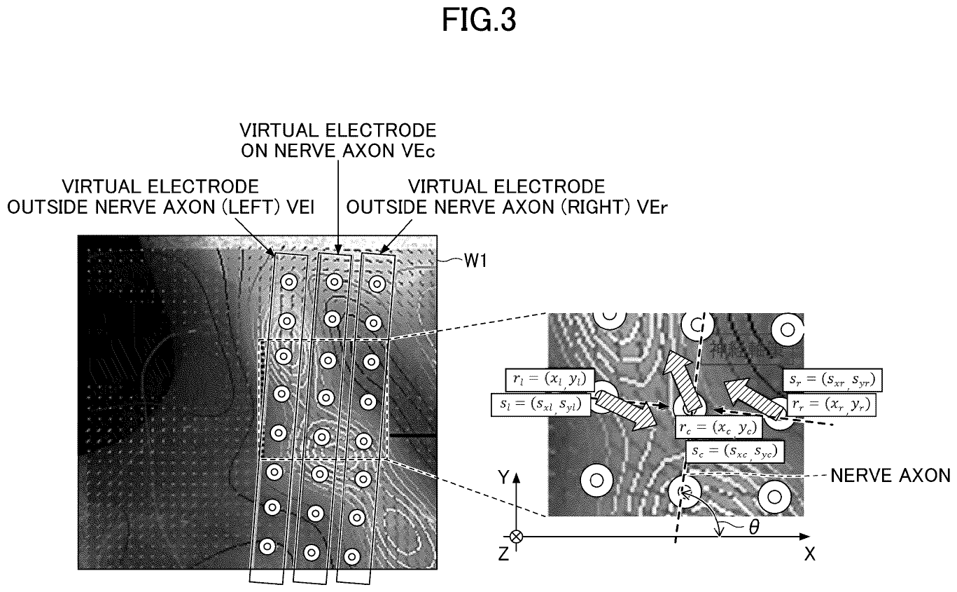

In the data processing device 30, the input control part 40 includes a location input part 41 and a waveform region designation part 42, and performs input processing of various kinds of information input from an operator of the data processing device 30 via the input device 80 such as a mouse or a keyboard. The operator of the data processing device 30 may be an evaluator who evaluates a current waveform of a biological signal described later. The location input part 41 receives a location of a virtual electrode VE (FIG. 3) on the morphological image of the subject displayed on the display device 90 such as a liquid crystal display.

The waveform region designation part 42 causes a waveform display part 52 to display information indicating coordinates of a window for displaying the current waveform of the biological signal, for example, based on a window setting command received from the input device 80. The waveform display part 52 displays the current waveform of the biological signal or the like in a region surrounded by the indicated coordinates on the display screen of the display device 90.

The display control part 50 includes an image display part 51, the waveform display part 52, and a noise level display part 53, and performs control to display an X-ray image, a current waveform, a noise level, and the like on the display device 90. The functions of the image display part 51, the waveform display part 52, and the noise level display part 53 will be described with reference to FIG. 3 and the like. Note that the input device 80 and the display device 90 may be included in the data processing device 30. An output device such as a printer may be connected to the data processing device 30.

The operation control part 60 includes a pathway generation part 61, a virtual electrode generation part 62, a reconstruction analysis part 63, a current component extraction part 64, and a noise level calculation part 65. The functions of the pathway generation part 61, the virtual electrode generation part 62, the reconstruction analysis part 63, the current component extraction part 64, and the noise level calculation part 65 will be described with reference to FIG. 3 and the like.

The storage part 70 is achieved by a storage device such as a hard disk drive (HDD), for example, and has an area for storing biomagnetic data 71, morphological image data 72, noise level data 73, and analysis parameter 74 for one or more analysis parameters of various types. The biomagnetic data 71 includes magnetic measurement data which is measured by the magnetic sensor part 10 and processed by the signal acquisition part 20. The morphological image data 72 includes either one of X-ray image data captured by an X-ray imaging device (not illustrated) and magnetic resonance (MR) image data captured by a magnetic resonance tomography device (not illustrated) or both. In the following description, an X-ray morphological image of a subject generated from X-ray image data is referred to as an X-ray image, and a cross-sectional morphological image of a subject generated from MR image data is referred to as an MR image.

The noise level data 73 indicates noise levels for each virtual electrode VE calculated by the noise level calculation part 65. The analysis parameter 74 includes parameters necessary for measuring biomagnetism, such as setting values of filters (a high pass filter and a low pass filter) provided in the signal acquisition part 20, a time range in which various signal processes are performed, and number of elements, and parameters necessary for performing the biometric information display method. The analysis parameter 74 may include a calculation method, a calculation formula, or the like used when calculating the noise level described below, and graphic representation data including a graphic representation representing the noise level described below.

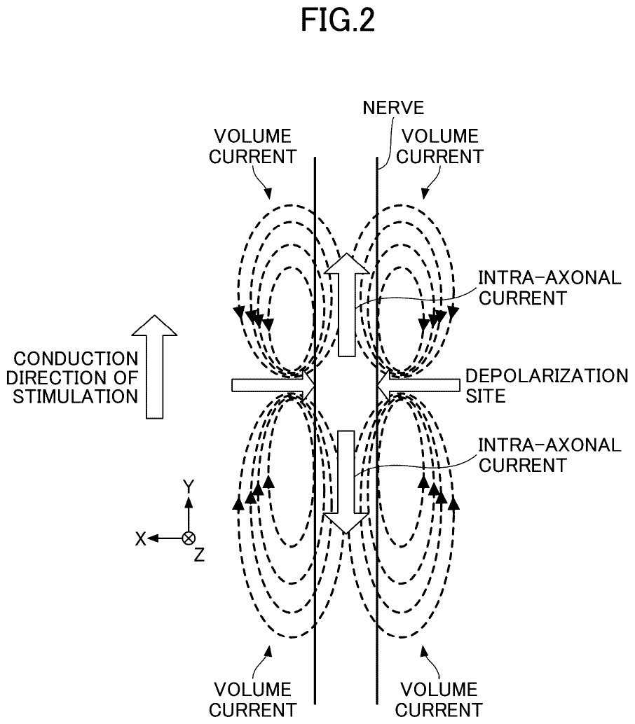

FIG. 2 is a diagram that explains an example model of nerve action currents. FIG. 2 illustrates how currents are generated from the action of a nerve that runs vertically straight in the drawing. The lower side of the drawing is the peripheral nerve side and the upper side of the drawing is the central nerve side. For example, when an electrical stimulation is applied to a peripheral nerve, the stimulation produces currents that travel through the nerve axon from the lower side to the upper side.

At this time, an intra-axonal current that flows to the upper side (forward direction) of the drawing, an intra-axonal current that flows to the lower side (reverse direction) of the drawing, and volume currents, which are current components that flow outside the nerve axon and return to the depolarization site, are produced. In order to evaluate the nerve function in detail, it is preferable to extract the intra-axonal currents that flow along the nerve axon and the currents that flow perpendicularly into the nerve axon due to the volume currents to visually display the extracted currents on the screen of the display device 90 or the like.

FIG. 3 is a diagram that explains an example of virtual electrodes and bioelectric currents displayed in a superimposed manner on a morphological image of a living body. Virtual electrodes VEc, VEl, and VEr and the bioelectric currents can be superimposed over the morphological image by the data processing device 30 of FIG. 1. The arrangement of the virtual electrodes VEc, the virtual electrodes VEl, and the virtual electrodes VEr are examples of measurement locations. Hereinafter, the virtual electrodes VEc, VEl, and VEr are also referred to as virtual electrodes VE in a case where the virtual electrodes VEc, VEl, and VEr are not distinguished from each other.

The virtual electrode VE is a virtual electrode set on an X-ray image or an MR image of a subject, and indicates a current extraction point (a current observation point) for extracting (reconstructing) a current component from magnetic data among magnetic measurement portions of the subject. The method of extracting a current component is known, and therefore, detailed description thereof is omitted.

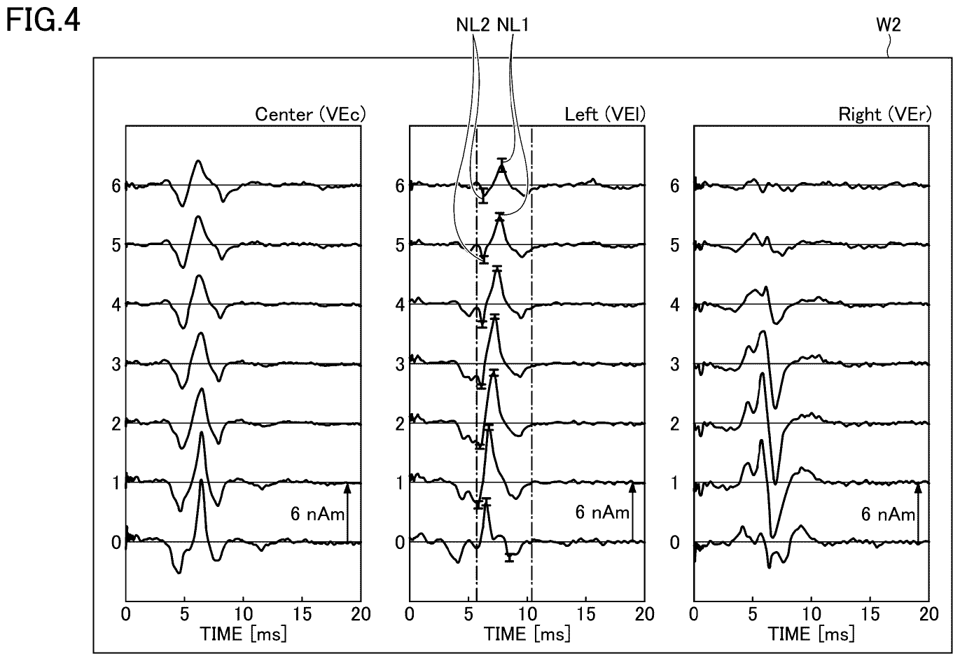

Note that the X-Y plane illustrated in FIG. 3 is a plane parallel to a detection surface of a SQUID magnetic sensor array. The X direction is a direction in which the SQUID magnetic sensor array is arranged, the Y direction is a direction orthogonal to the X direction, and the Z direction is a direction orthogonal to the X-Y plane and is a direction facing the SQUID magnetic sensor array. The reference codes r_{c}, s_{c}, r₁, s₁, rᵣ, and sᵣ illustrated in FIG. 4 indicate the following:
r_{c}: intra-axonal current acquisition location=(x_{c}, y_{c})
s_{c}: estimated current at r_{c}=(s_{xc}, s_{yc})
rₗ: left volume current acquisition location=(xₗ, yₗ)
sₗ: estimated current at rₗ=(sₓₗ, s_{yl})
rᵣ: right volume current acquisition location=(xᵣ, yᵣ)
sᵣ: estimated current at rᵣ=(sₓᵣ, s_{yr})
θ: angle of nerve axon (=angle in a nerve axonal direction with respect to X direction)

In FIG. 3, in the morphological image (X-ray image) of the subject, the virtual electrodes VEc are placed on a nerve pathway (nerve axon) that runs linearly. The virtual electrodes VEl and VEr outside the nerve axon (on the left side and the right side) are provided on the left side and the right side of the virtual electrode VEc on the nerve axon (in the direction orthogonal to the nerve pathway), respectively. In a display window W1 of the morphological image illustrated in FIG. 3, a rectangle around each set of the virtual electrodes VEc, VEl, and VEr is provided for the sake of explanation, and is not displayed on the actual screen. The display window W1 is displayed on the screen of the display device 90.

Although it is difficult to ascertain in FIG. 3, small arrows that are superimposed over the image in the display window W1 indicate the directions of the currents extracted by the configuration, and the lengths of the arrows indicate the current intensity. Note that the opposite end of the arrowhead indicates the location of a voxel which is a unit of extraction of a current component. The contour-like curve is a current intensity distribution line generated by connecting locations at which the current intensity is the same.

The nerve pathway passing through the nerve axon is generated by the location input part 41 detecting locations designated on the morphological image through the input device 80 and the pathway generation part 61 performing calculation using coordinates of the detected locations. For example, the location indicating the pathway of the nerve axon is input from the input device 80 by the operator who operates the data processing device 30 by observing the nerve in the morphological image.

Alternatively, the pathway of the nerve axon may be set by the data processing device 30 inferring the location, the running direction, and the like of the nerve in the morphological image. In a case where the pathway of the neural axon is set by inference, machine learning such as deep learning is performed in advance with a large number of morphological images in which pathways are known, and a neural network for setting a pathway is constructed by the pathway generation part 61 of the data processing device 30. The neural network for setting a pathway may be constructed in a cloud connected to the data processing device 30 via a network and used by the data processing device 30.

In FIG. 1, the pathway generation part 61 extracts a pathway such as a nerve pathway by generating a curve (including a straight line), based on the coordinates of the locations on an image, the coordinates being designated by the operator observing the morphological image displayed on the display device 90, for example. The pathway generation part 61 notifies the virtual electrode generation part 62 of information indicating the extracted pathway. The extracted pathway is presented by coordinate information, an equation that represents the curve, or the like.

The virtual electrode generation part 62 generates the virtual electrodes VEc at equal intervals, for example, on the pathway notified from the pathway generation part 61. The interval and the number of virtual electrodes VEc generated on the pathway are designated by the operator through the input device 80 and stored in the storage part 70 by the input control part 40. The virtual electrode generation part 62 generates the virtual electrodes VEc on the pathway generated by the pathway generation part 61 in accordance with the intervals and the number stored in the storage part 70.

The virtual electrode generation part 62 also sets a virtual electrode VEl (on the left side) and a virtual electrode VEr (on the right side) outside the nerve axon on the left side and the right side of the virtual electrode VEc, respectively. The virtual electrode generation part 62 stores the set coordinates of the virtual electrodes VEc, VEl, and VEr on the image in the storage part 70.

The distance from the virtual electrode VEc to the virtual electrodes VEl and VEr outside the nerve axon may be designated by the operator through the input device 80, similarly to the interval between the virtual electrodes VEc. In a case where the storage part 70 does not store any one of information indicating the interval, the number, and the distance of the virtual electrodes VE, the virtual electrode generation part 62 generates the virtual electrodes VE using a preset default value.

The reconstruction analysis part 63 operates independently of the operations of the pathway generation part 61 and the virtual electrode generation part 62, and reconstructs current components for each voxel, which is a virtual current extraction point arranged at a predetermined interval, by using the magnetic data of the subject measured by the magnetic sensor part 10. The reconstruction analysis part 63 stores, in the storage part 70, current information indicating the direction, intensity, coordinates, and the like of the current for each voxel obtained by the reconstruction. The reconstruction analysis part 63 causes the image display part 51 to superimpose arrows each indicating the direction and intensity of the current for a voxel obtained by the reconstruction over the morphological image and display the superimposed image.

The reconstruction analysis part 63 also extracts locations having the same current intensity on the image based on the current intensity of each voxel. The reconstruction analysis part 63 then stores a line connecting the extracted locations in the storage part 70 as a current intensity distribution line, and causes the image display part 51 to display the current intensity distribution line with superposition on the morphological image. As a result, as illustrated in FIG. 3, the current distribution reconstructed for each measurement period (the arrows of the currents and the current intensity distribution lines) are superimposed over the morphological image and the like, and the superimposed image is displayed on the screen.

Based on a location relationship between each of the virtual electrodes VEc, VEl, and VEr and voxels, the current component extraction part 64 extracts the current component (current density distribution) of each virtual electrode VE by using the current components at the voxels. The current component extraction part 64 acquires current data that changes over time as a current waveform at each of the virtual electrodes VEc, VEl, and VEr to store the acquired current waveforms (time-series data of the current components) in the storage part 70. The current component extraction part 64 causes the waveform display part 52 to display the acquired current waveforms on the screen of the display device 90. A display example of the current waveforms is illustrated in FIG. 4. In the following description, displaying on the screen of the display device 90 is also referred to as displaying on the display device 90.

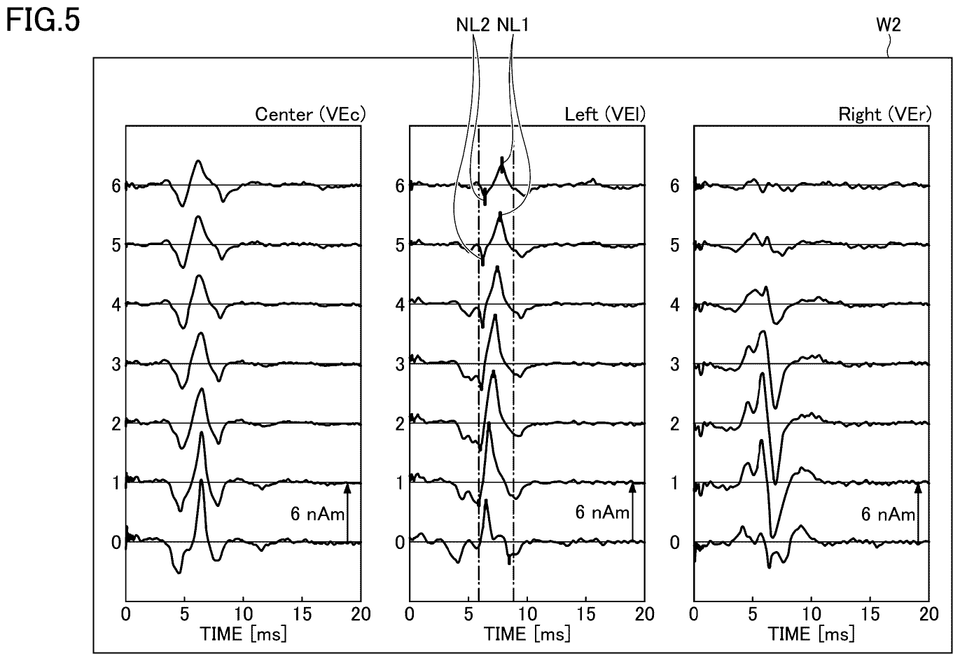

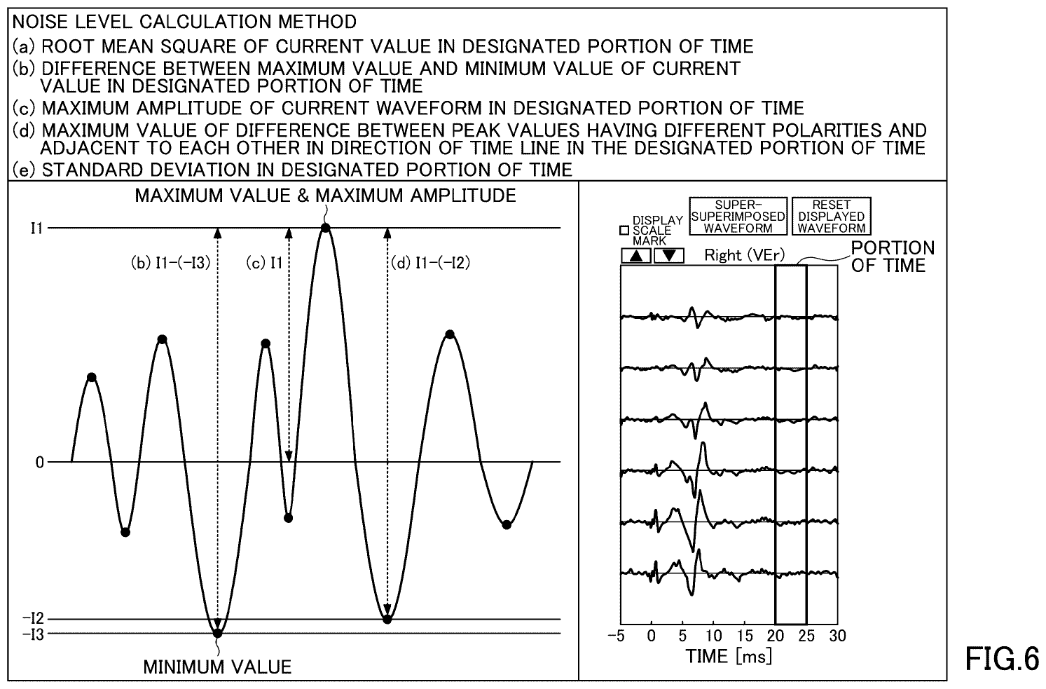

The noise level calculation part 65 calculates the noise level for each virtual electrode VE, based on the time-series data of the current component (biological current) which has been extracted for each virtual electrode VE by the current component extraction part 64 and stored in the storage part 70. A method of calculating the noise level will be described with reference to FIG. 6. The noise level calculation part 65 stores the calculated noise levels for each virtual electrode VE in the storage part 70 as the noise level data 73. The noise level calculation part 65 then causes the noise level display part 53 to display, on the display device 90, a graphic representation visually representing the noise level by superimposing the noise level over a peak of the current waveform of each virtual electrode VE, for example. Examples of the graphic representation representing the noise level are illustrated in FIG. 4 and FIG. 5.

Note that the noise level calculation part 65 may display the noise level on the display screen at the same reduced scale as the scale used for the current waveform, or may display the noise level on the display screen at a reduced scale larger than that used for the current waveform. Furthermore, the noise level calculation part 65 may calculate a noise level included in the current waveform that the current component extraction part 64 has acquired in the past and stored in the storage part 70. In such a case, the noise level calculation part 65 may display the noise level of the current waveform acquired in the past on the display screen on which both the present current waveform and the noise level of the present current waveform are displayed.

As illustrated in FIG. 3, the image display part 51 displays the morphological image on the display device 90, and also displays, the virtual electrodes VEc, VEl, and VEr, the arrows each indicating the direction and the intensity of the current for each voxel, and current intensity distribution lines superimposed over the morphological image on the display device. The waveform display part 52 displays the current waveform for each virtual electrode VE on the display device 90. The noise level display part 53 displays, on the display device 90, the graphic representation representing the noise level included in the current waveform of the virtual electrode VE by superimposing the graphic representation over the peak of the current waveform of the virtual electrode VE, using the noise level data 73 calculated by the noise level calculation part 65 and stored in the storage part 70.

FIG. 4 is a diagram that explains an example of current waveforms and graphic representations representing noise levels displayed on the display device 90 of FIG. 1. A display window W2 illustrated in FIG. 4 is displayed on the screen of the display device 90, for example, side by side with the display window W1 in which the morphological image illustrated in FIG. 3 is displayed. The current waveforms of the virtual electrodes VEc, VEl, and VEr are displayed in the display window W2. Numbers illustrated next to the current waveforms of the virtual electrodes VEc, VEl, and VEr indicate electrode numbers.

The current waveform (changes of current intensity over time) is displayed for each of the virtual electrodes VEc, VEl, and VEr, with a horizontal axis representing time common to all the electrode numbers. A numerical value (“6 nAm” in this example) illustrated at a lower right part of each graph in FIG. 4 indicates a current dipole. In the display window W2, the current waveforms at the virtual electrodes VEc, VEl, and VEr in FIG. 3 are displayed by the waveform display part 52.

The change in the currents that flow through the nerve axon can be seen from the current waveforms of the virtual electrode VEc. The change in the volume currents that flow into the nerve axon can be seen from the current waveforms of the virtual electrodes VEl and VEr. For example, the current is evaluated by latency, which is a time it takes, after an electric stimulation is applied to a peripheral nerve of the subject, for the current value to change significantly. The latency is detected by a peak value of the current waveform.

When evaluating whether the nerve action is normal, for example, the current waveform of the virtual electrode VEc indicating the current that flows through the nerve axon and the current waveforms of the virtual electrodes VEl and VEr indicating the currents that flow into the nerve axon are referred to. Through the change in the current waveform of each of the virtual electrodes VEc, VEl, and VEr, it can be confirmed that the peak latency of the nerve action is advanced or not.

In the display window W2, I-shaped graphic representations representing noise levels NL1 and NL2 are displayed such that the noise levels are superimposed over the respective peaks of the current waveform of the virtual electrode VEl by the noise level display part 53. For example, in the I-shaped graphic representation, the length in the direction orthogonal to a direction representing time indicates the magnitude of the noise level set to the same reduced scale as that of the current intensity of the current waveform.

In FIG. 4, the noise levels NL1 and NL2 are calculated for each virtual electrode VEl to be displayed as graphic representations; however, the I-shaped graphic representations representing the noise levels NL1 and NL2 of one given virtual electrode VEl may be displayed as common noise levels of the other virtual electrodes VEl.

In FIG. 4, the graphic representations representing the noise levels NL1 and NL2 are superimposed over the current waveforms of only the virtual electrodes VEl; however, the graphic representations representing the noise levels may be superimposed over one or more of the current waveforms of the virtual electrodes VEc, VEl, and VEr. The evaluator can select the current waveforms of which of the virtual electrodes VEc, VEl, and VEr the graphic representation representing the noise levels are to be superimposed over by operating the input device 80 to select the virtual electrode from a selection window displayed on the display screen of the display device 90.

The graphic representation representing the noise level NL1 is displayed corresponding to a positive peak of the current waveform of each virtual electrode VEl. The graphic representation representing the noise level NL2 is displayed corresponding to a negative peak of the current waveform of each virtual electrode VEl. In the example illustrated in FIG. 4, for example, the evaluator who evaluates the current waveform displayed in the display window W2 evaluates the state of the subject by the positive peaks of the current waveforms of the respective virtual electrodes VEl. The evaluator therefore does not determine whether or not the current waveform is usable for assisting the evaluation of the nerve action, based on the graphic representation representing the noise level NL2 superimposed over the negative peak of the current waveform.

When evaluating the nerve action, whether the amplitude of the peak of the current waveform is reduced or not is important. For example, when the current waveform indicating the conduction of the nerve action is affected by noise, the evaluation of the amplitude of the current waveform and the evaluation of the peak latency are affected, and thus the nerve action may not be normally evaluated. Therefore, in order to intuitively determine the current waveform that cannot be used for evaluation, it is important to visually display the degree of influence of noise on the current waveform.

For example, in FIG. 4, the I-shaped graphic representation each representing the noise levels NL1 and NL2 indicates the magnitude of the noise level set to the same reduced scale as that of the current intensity of the current waveform. Each I-shaped graphic representation is displayed with the center of the I-shaped graphic representation superimposed over the location of the respective peaks of the current waveform. By superimposing the graphic representation representing the noise level on the same reduced scale as that of the current intensity on the current waveform, the evaluator can intuitively ascertain a SN ratio which is a ratio between the signal level and the noise level included in the current waveform. Thus, the evaluator can intuitively ascertain to what extent the current waveform of the virtual electrode VEl of interest is influenced by noise.

As a result, the evaluator can easily and appropriately determine whether or not the acquired current waveform is a current waveform that can be used to assist in the evaluation of the nerve action. For example, it is possible to intuitively ascertain a current waveform having a small SN ratio and determine not to use the current waveform for assisting evaluation. In a case where biomagnetic data is measured under the same measurement conditions and the reproducibility of the current waveform is examined by superimposing the current waveform of the bioelectric current calculated from the magnetic data, it is possible to easily visually determine whether the acquired waveform is a valid current waveform.

Note that the noise level display part 53 may display the noise level on the display device 90 at a reduced scale larger than that of the current waveform (for example, twice or three times). In FIG. 4, the graphic representations representing the noise levels NL1 and NL2 are emphasized; however, the graphic representations representing the noise levels NL1 and NL2 may have the same thickness as that of the current waveform or may be displayed in a color different from that of the current waveform. Furthermore, the waveform display part 52 may display the current waveform including the noise level of equal to or higher than a preset threshold value in a color different from that of the current waveform including the noise level of lower than the threshold value. In such a case, the current waveforms each including the noise level of equal to or higher than the threshold value can be easily recognized.

FIG. 5 is a diagram that explains another example of the graphic representation representing the noise level displayed on the display device. In the example illustrated in FIG. 5, the noise level display part 53 displays a line-shaped graphic representation which is oriented in a direction orthogonal to an axis representing time and whose center intersects the current waveform, instead of the I-shaped graphic representation illustrated in FIG. 4. The length of the line-shaped graphic representation indicates the magnitude of the noise level set to the same reduced scale as that of the current intensity of the current waveform. Note that the noise level display part 53 may display the line-shaped graphic representations illustrated in FIG. 5 at all of the times of the current waveform. In such a case, a band-like graphic representation representing the noise levels is displayed along the current waveform. The graphic representation representing the noise level may be a rectangle or an arrow.

FIG. 6 is a diagram that explains an example of a method of calculating the noise level illustrated in FIG. 4. The noise level calculation part 65 of FIG. 1 calculates the noise level included in the current waveform for a designated portion of time during the measurement period in which the biological signal is measured. For example, the measurement period in which the biological signal is measured is indicated by the time displayed in the display window W2, and is 35 ms from −5 ms to 30 ms in FIG. 6. The 0 ms is a timing at which an electric stimulation is applied to the peripheral nerve, for example.

In the example illustrated in FIG. 6, the designated portion of time is a region of 5 ms from 20 ms to 25 ms. The portion of time may be set, in the measurement period, to a time period during which a biological signal (current waveform) of interest does not appear. For example, the portion of time may be set to 5 ms from −5 ms to 0 ms before an electric stimulation is applied.

The evaluator who evaluates the current waveform can designate the portion of time by operating an input window displayed on the display device 90 through the input device 80. The evaluator can also select a method of calculating the noise level by operating an input window displayed on the display device 90 through the input device 80. The portion of time for the noise level calculation may be included in the measurement period that is not displayed in the display window W2.

In the example illustrated in FIG. 6, the evaluator can select any one of five calculation methods illustrated in (a) to (e). In a case where the method (a) is selected, the noise level calculation part 65 calculates a root mean square (RMS) of the current value in the designated portion of time. In a case where the method (b) is selected, the noise level calculation part 65 calculates a difference (I1-(−I3)) between the maximum value (I1) and the minimum value (−I3) of the current value in the designated portion of time.

In a case where the method (c) is selected, the noise level calculation part 65 calculates the maximum amplitude (I1) of the current waveform in the designated portion of time. In a case where the method (d) is selected, the noise level calculation part 65 calculates the maximum value (I1-(−I2)) of the difference between peak values having different polarities and being adjacent to each other in the axis representing time in the designated portion of time. In a case where the method (e) is selected, the noise level calculation part 65 calculates a standard deviation of the current value in the designated portion of time.

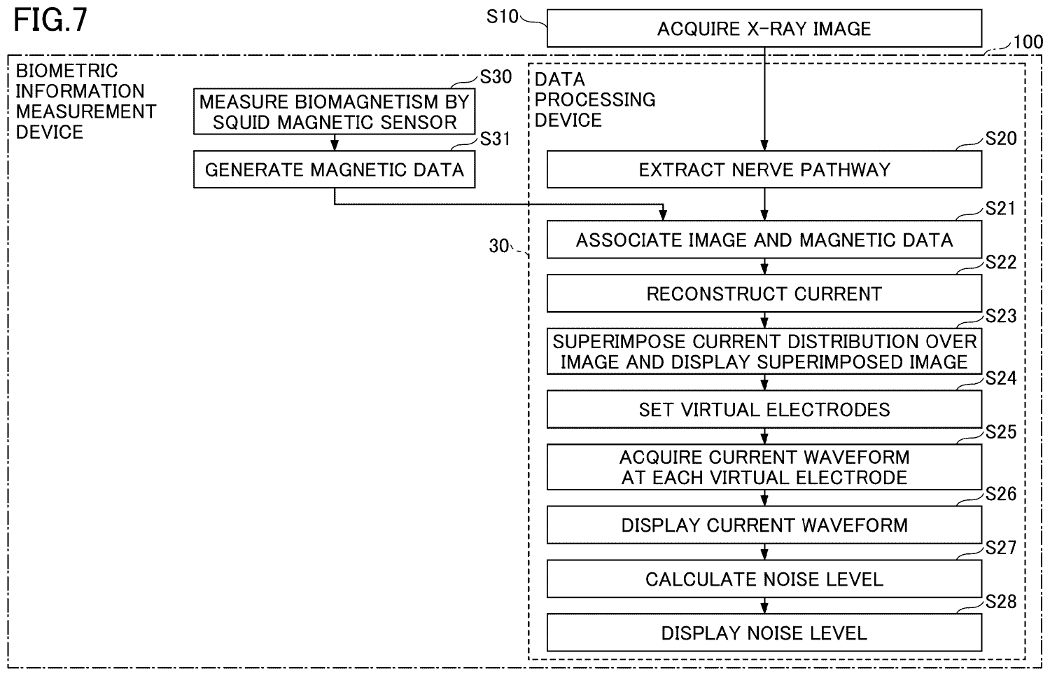

FIG. 7 is a flowchart illustrating an example of processing for displaying the current waveform and the graphic representation representing the noise level on the display device 90 by the data processing device 30 of FIG. 1. The flow in FIG. 7 illustrates the processing of the biometric information display method for displaying the biometric information on the display device 90, and is achieved by the biometric information display program executed by the data processing device 30.

In the example illustrated in FIG. 7, in order to acquire a morphological image of a measurement-target part of the subject, an X-ray image of the subject is acquired using the X-ray imaging device in step S10 separately from the measurement of the magnetic signal of the subject, and is stored, as the morphological image data 72, in the storage part 70. The image display part 51 displays, on the display device 90, a morphological image of the morphological image data 72 stored in the storage part 70.

Thereafter, in step S20, the pathway generation part 61 of the data processing device 30 generates a curve (including a straight line) based on the coordinates of the locations on the image designated by the operator by observing the morphological image displayed on the display device 90, thereby extracting a nerve pathway. The extraction of the nerve pathway may be automatically performed by machine learning such as deep learning.

The image display part 51 displays the nerve pathway, which has been extracted by the pathway generation part 61, by superimposing the nerve pathway over the morphological image displayed on the screen of the display device 90. The pathway generation part 61 stores the coordinates of the extracted nerve pathway on the image, an equation that represents the curve, and the like in the storage part 70.

In step S30, the biometric information measurement device 100 measures biomagnetism of the subject by the SQUID magnetic sensor in synchronization with the electrical pulse stimulation applied to the peripheral nerve of the subject, for example. In step S31, the signal acquisition part 20 generates digital magnetic data based on the voltage output by the SQUID magnetic sensor in response to the measured magnetism, and stores the generated magnetic data, as the biomagnetic data 71, in the storage part 70. Note that steps S30 and S31 are repeatedly performed. By this point, the preparation for acquiring the current waveform of the measurement-target part of the subject is completed.

In step S21 after step S20, the operation control part 60 of the data processing device 30 associates the measurement locations of the magnetic data with the morphological image from which the nerve pathway has been extracted in step S20. Next, in step S22, the reconstruction analysis part 63 reconstructs current components based on the magnetic data for each measurement period. The reconstruction analysis part 63 stores, as the morphological image data 72, current information indicating the directions, intensity, coordinates, and the like of the currents at the reconstruction points obtained by the reconstruction in the storage part 70.

Next, in step S23, the reconstruction analysis part 63 causes the image display part 51 to display the arrows indicating the directions and intensity of currents at the reconstruction points obtained by the reconstruction on the morphological image. The reconstruction analysis part 63 extracts locations having the same current intensity on the image based on the current intensity at each reconstruction point, and displays the extracted locations, as the current intensity distribution line which connects the extracted locations, by superposing on the morphological image. The reconstruction analysis part 63 stores, as the morphological image data 72, information indicating the current intensity distribution line in the storage part 70. Thus, for example, as illustrated in FIG. 3, the X-ray image and the like and the distribution of the current reconstructed for each measurement period (the arrows of the current and the current intensity distribution lines) are displayed superposed on the screen.

In step S24, the virtual electrode generation part 62 sets a predetermined number of virtual electrodes VEc at intervals on the nerve pathway extracted in step S20. The number and the interval of the virtual electrodes VEc are set in advance by the operator or the like. The virtual electrode generation part 62 also sets virtual electrodes VEl and VEr on the right side and the left side of each virtual electrode VEc, respectively, in the direction orthogonal to the nerve pathway. The virtual electrode generation part 62 stores, as the morphological image data 72, the set coordinates of the virtual electrodes VEc, VEl, and VEr on the image in the storage part 70.

The virtual electrode generation part 62 causes the image display part 51 to display the virtual electrodes VEc, VEl, and VEr which have been set by superimposing the electrodes over the morphological image in which the nerve pathway is displayed. As a result, as illustrated in FIG. 3, the virtual electrodes VEc set on the nerve pathway (nerve axon), the virtual electrodes VEl and VEr set on the right side and the left side of each virtual electrode VEc, respectively, and the like are superimposed over the X-ray image or the like and displayed on the display device 90.

Next, in step S25, the current component extraction part 64 acquires, as a current waveform, current data that changes over time at each of the virtual electrodes VEc, VEl, and VEr. In step S26, the current component extraction part 64 causes the waveform display part 52 to display the acquired current waveform on the display screen of the display device 90. The process of step S26 is an example of a waveform display process of displaying a biological signal waveform including the measured biological signal.

The waveform display part 52 displays, on the display device 90, changes of the current component extracted for each virtual electrode VE over time by the current component extraction part 64 as a current waveform. For example, the current waveform is displayed in the display window W2 illustrated in FIG. 4 or 5, which is different from the display window W1 in which the morphological image illustrated in FIG. 3 is displayed on the screen of the display device 90.

Next, in step S27, the noise level calculation part 65 calculates the noise level in a preset portion of time by using a preset calculation method. The noise level calculation part 65 stores the calculated noise levels in the storage part 70 as the noise level data 73.

Next, in step S28, the noise level calculation part 65 causes the noise level display part 53 to display, on the display screen of the display device 90, the graphic representation representing the noise level corresponding to the virtual electrode VE designated to be displayed, among the calculated noise levels. At this time, the noise level calculation part 65 causes the noise level display part 53 to acquire the graphic representation data of the graphic representation to be displayed from the analysis parameter 74. The process of step S28 is an example of a noise level display process of displaying the graphic representation representing the noise level included in the current waveform at a location corresponding to the peak of the current waveform.

The noise level display part 53 acquires the designated graphic representation data from the analysis parameter 74. The noise level display part 53 then displays the graphic representation representing the noise level corresponding to the designated virtual electrode VE at a location corresponding to the peak of the current waveform of the designated virtual electrode VE. Thus, as illustrated in FIG. 4, a series of processes of displaying the current waveform and the graphic representation representing the noise level in the display window W2 is completed.

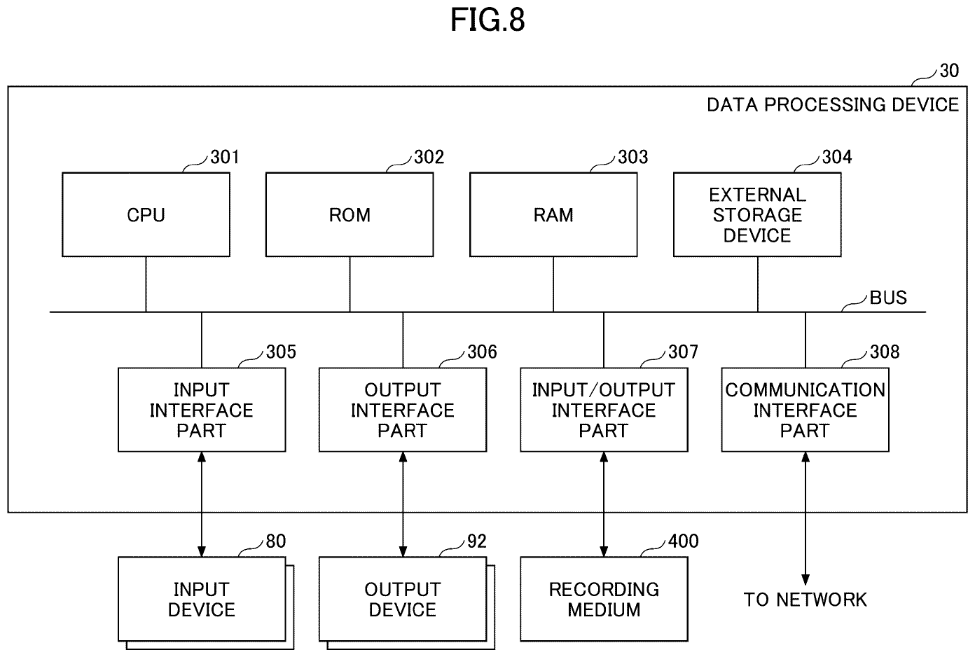

FIG. 8 is a block diagram illustrating an example of a hardware configuration of the data processing device 30 illustrated in FIG. 1. The data processing device 30 includes a CPU 301, a read only memory (ROM) 302, a random access memory (RAM) 303, and an external storage device 304. The data processing device 30 also includes an input interface part 305, an output interface part 306, an input/output interface part 307, and a communication interface part 308. For example, the CPU 301, the ROM 302, the RAM 303, the external storage device 304, the input interface part 305, the output interface part 306, the input/output interface part 307, and the communication interface part 308 are interconnected via a bus BUS.

The CPU 301 executes various programs such as an operating system (OS) and applications, and controls the overall operation of the data processing device 30. The ROM 302 holds basic programs, various parameters, and the like for enabling the CPU 301 to execute various programs. The RAM 303 stores the various programs executed by the CPU 301, data used in the programs, and the like. The various programs include a morphological image display program for displaying the morphological image illustrated in FIG. 3 and a current waveform display program for displaying the current waveforms and the graphic representations representing the noise levels illustrated in FIG. 4. The morphological image display program and the current waveform display program are examples of a biometric information display program.

The external storage device 304 is a hard disk drive (HDD), a solid state drive (SSD), or the like, and stores various programs to be loaded into the RAM 303. The various programs may include a noise level calculation program for calculating a noise level and a current waveform display program for displaying the current waveform reconstructed from magnetic data and the graphic representation representing the noise level on the display device 90.

The input interface part 305 is connected with the input device 80, such as a keyboard, a mouse, or a tablet, that receives input from the operator or the like who operates the data processing device 30. The output interface part 306 is connected with an output device 92 (for example, the display device 90 in FIG. 1) such as a display device for displaying a display screen or the like generated by various programs executed by the CPU 301 or a printer.

A recording medium 400 such as a universal serial bus (USB) memory is connected to the input/output interface part 307. For example, the recording medium 400 may store various programs such as the above-described morphological image display program and the current waveform display program. In such a case, the programs are transferred from the recording medium 400 to the RAM 303 through the input/output interface part 307. Note that the recording medium 400 may be a CD-ROM, a digital versatile disc (DVD: registered trademark), or the like. In such a case, the input/output interface part 307 has an interface supporting the recording medium 400 to be connected. The communication interface part 308 connects the data processing device 30 to a network or the like.

As described above, in the first embodiment, it is possible for a user to intuitively ascertain a degree of the influence of noise on a current waveform of a biological signal by displaying a graphic representation representing a noise level included in the current waveform of the biological signal at a location corresponding to a peak of the current waveform. By setting the graphic representation representing the noise level to have a length corresponding to the magnitude of the noise level, the SN ratio, which is a ratio between the signal level and the noise level included in the current waveform, can be intuitively ascertained.

When calculating the noise level in a designated portion of time during a measurement period in which the biological signal is measured, the evaluator can designate an appropriate portion of time by observing the current waveform displayed in the display window W2. For example, by setting the portion of time to a time period during which the biological signal of interest does not appear, a more accurate noise level can be calculated.

By making the color of the current waveform including the noise level of equal to or higher than a preset threshold value different from the color of the current waveform including the noise level of lower than the threshold value, the current waveform including the noise level of equal to or higher than the threshold value can be easily recognized. Thus, it is possible to appropriately identify whether or not the current waveform is usable for assisting evaluation of the nerve action.

### Second Embodiment

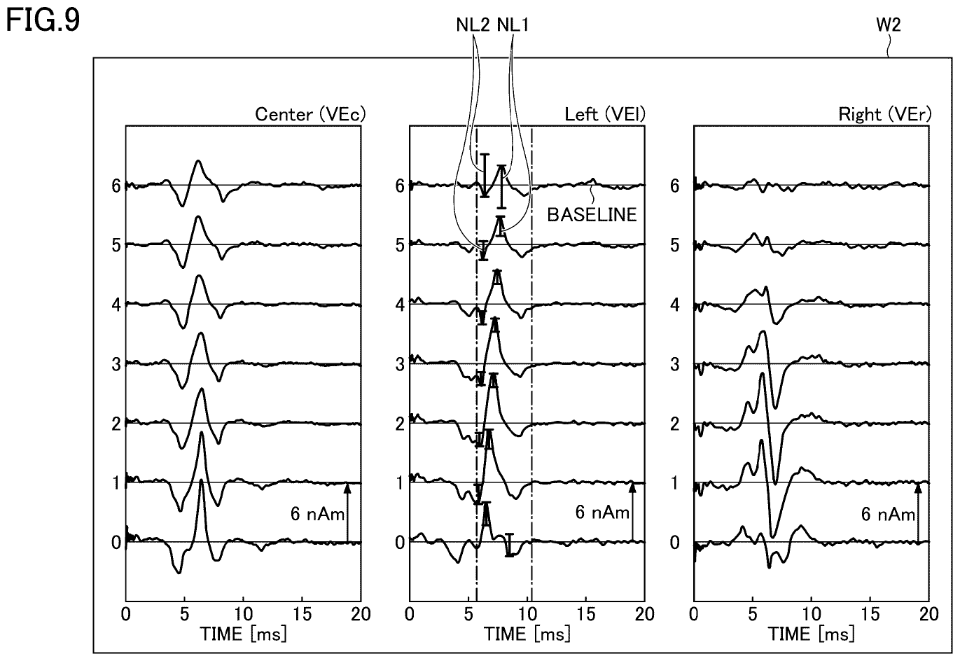

FIG. 9 is a diagram that explains an example of current waveforms and graphic representations representing noise levels displayed on the display device in a second embodiment of the present disclosure. Detailed description of the same elements and functions as those in the first embodiment will be omitted. In this embodiment, a configuration and functions of a biometric information measurement device equipped with a display device are the same as the configuration and functions of the biometric information measurement device 100 illustrated in FIG. 1 except that the function of the noise level display part 53 is different.

By multiplying the noise level for each virtual electrode VE which is calculated by the noise level calculation part 65 by n (n is a value larger than 1), the noise level display part 53 of the present embodiment displays the I-shaped graphic representation representing the multiplied noise level in the display window W2. For example, the noise level display part 53 triples the noise level (n=3). When displaying the I-shaped graphic representation representing the multiplied noise level, the noise level display part 53 displays the graphic representation with one end at the location corresponding to the peak of the current waveform and the other end extending toward a baseline which is displayed parallel to the axis representing time.

Note that in FIG. 9, the state of the subject is evaluated by the positive peak of the current waveform of each virtual electrode VEl, as in FIG. 4. For example, the evaluator determines that the current waveform in which the I-shaped graphic representation representing the noise level NL1 crosses the baseline cannot be used for the evaluation. That is, the graphic representation representing the noise level multiplied by n is used as a threshold value for determining whether or not the evaluation is usable. In such a manner, the evaluator can intuitively ascertain the degree of the influence of noise on the current waveform of the virtual electrode VEl of interest by the graphic representation representing the noise level multiplied by n.

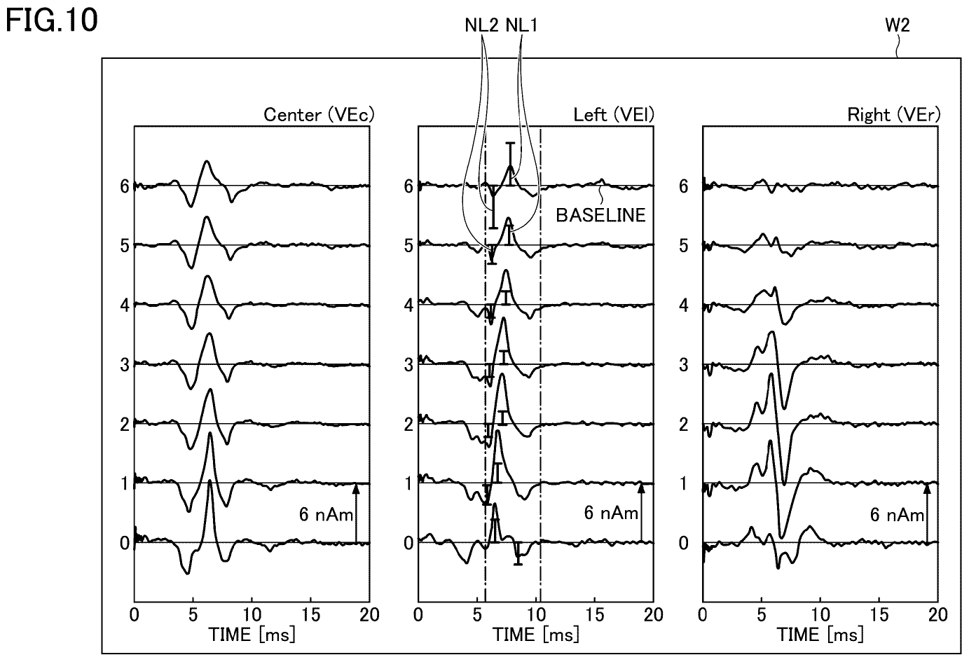

FIG. 10 is a diagram that explains yet another example of the graphic representation representing the noise level displayed on the display device. In FIG. 10, by multiplying the noise level for each virtual electrode VE calculated by the noise level calculation part 65 by n (three times, for example), the noise level display part 53 displays the I-shaped graphic representation representing the multiplied noise level in the display window W2. However, in FIG. 10, when displaying the I-shaped graphic representation representing the multiplied noise level, the noise level display part 53 displays the graphic representation with one end on the baseline and the other end extending toward the peak.

In FIG. 10, the state of the subject is evaluated by the positive peak of the current waveform of each virtual electrode VEl, as in FIG. 4. For example, when the I-shaped graphic representation straddles the current waveform, the evaluator of the current waveform determines that the current waveform cannot be used for the evaluation. That is, in FIG. 10, the graphic representation representing the noise level multiplied by n is used as a threshold value for determining whether or not to use the evaluation. In this way, the evaluator can intuitively ascertain the degree of the influence of noise on the current waveform of the virtual electrode VEl of interest by the graphic representation representing the noise level multiplied by n. Instead of the I-shaped graphic representation illustrated in FIG. 9 and FIG. 10, the line-shaped graphic representation illustrated in FIG. 5 or another bar-shaped graphic representation may be displayed. Furthermore, in the I-shaped graphic representation, a straight line parallel to the axis representing time may be displayed at the end part of the graphic representation on the opposite to the baseline. In such a case, only a straight line parallel to the axis representing time may be displayed without displaying the I-shaped graphic representation.

As described above, the second embodiment can also provide the same effects as those of the first embodiment. For example, the noise level display part 53 displays an I-shaped graphic representation obtained by multiplying the noise level by n on the display window W2 with one end at the location corresponding to the peak of the current waveform and the other end extending toward the baseline. Alternatively, the noise level display part 53 displays an I-shaped graphic representation obtained by multiplying the noise level by n in the display window W2 with one end on the baseline and the other end extending toward the peak. Thus, the evaluator can intuitively ascertain the degree of influence of noise on the current waveform of the virtual electrode VEl of interest by displaying the graphic representation representing the noise level which is multiplied by n.

### Third Embodiment

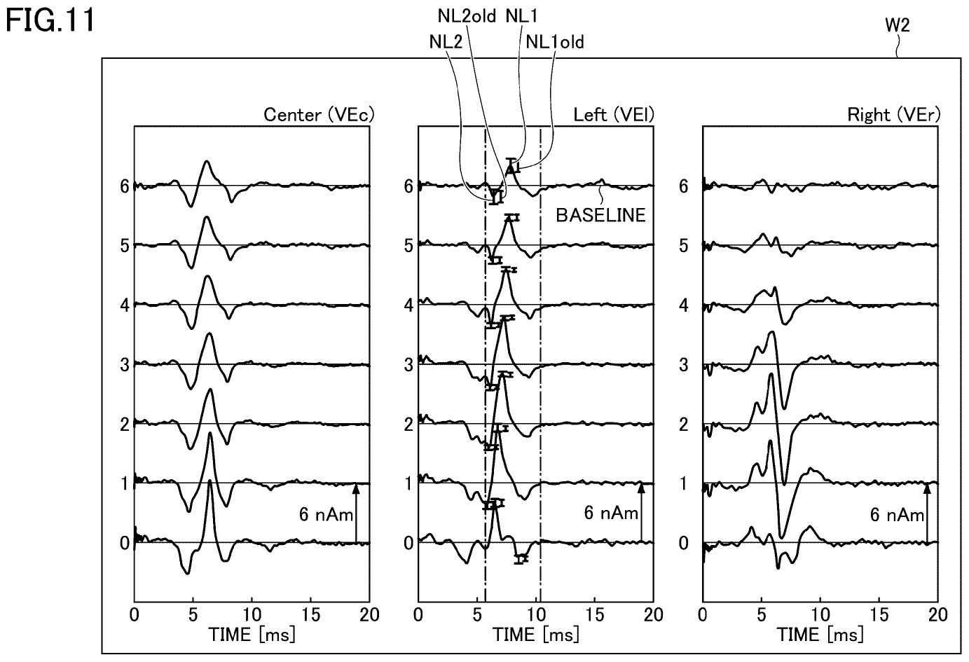

FIG. 11 is a diagram that explains an example of current waveforms and graphic representations representing noise levels displayed on the display device in a third embodiment of the present disclosure. Detailed description of the same elements and functions as those in the first embodiment will be omitted. In this embodiment, a configuration and functions of a biometric information measurement device equipped with a display device are the same as the configuration and functions of the biometric information measurement device 100 illustrated in FIG. 1 except that the function of the noise level display part 53 is different.

In this embodiment, the noise level calculation part 65 calculates each noise level included in the respective current waveforms calculated from the biomagnetism measured at different times, and stores the calculated noise levels in the storage part 70 as the noise level data 73.

The waveform display part 52 displays the designated current waveform and the graphic representations representing the noise levels NL1 and NL2 included in the current waveform in the display window W2, as in FIG. 4. Hereinafter, the designated current waveform is also referred to as a present current waveform. Furthermore, the waveform display part 52 displays graphic representations representing noise levels NL1old and NL2old included in a current waveform other than the present current waveform at present in the display window W2. For example, the current waveform including the noise levels NL1old and NL2old is a past current waveform calculated from the biomagnetism measured before the time the biomagnetism data from which the present current waveform displayed in the display window W2 is calculated is measured.

For example, by making the color of the graphic representations representing the noise levels NL1old and NL2old different from the color of the graphic representations representing the noise levels NL1 and NL2, the evaluator of the current waveform can easily identify the noise levels included in the current waveform at the present time and the current waveform in the past. The degree of the color of the graphic representations representing the noise levels NL1old and NL2old may be set lighter than that of the graphic representations representing the noise levels NL1 and NL2. The thickness of the graphic representations representing the noise levels NL1old and NL2old may be set to be thinner than that of the graphic representations representing the noise levels NL1 and NL2.

Furthermore, instead of displaying the I-shaped graphic representations, at locations away from the baseline by the lengths of the I-shaped graphic representations indicating the noise levels measured at different periods, respective straight lines may be displayed in parallel with the axis representing time. In such a case, for each noise level measured at the different period, the color or the line type of the straight line may be changed and displayed. Furthermore, the line-shaped graphic representation illustrated in FIG. 5 may be displayed instead of the I-shaped graphic representation illustrated in FIG. 11. The graphic representations representing the noise levels NL1, NL2, NL1old, and NL2old may represent noise levels multiplied by n as illustrated in FIG. 9 and FIG. 10.

By displaying the graphic representation representing the noise level of the past current waveform, it is possible to intuitively determine whether the noise included in the present current waveform is relatively large. Note that the noise level of the past current waveform may be an average value or the maximum value of the noise levels of the past current waveforms.

As described above, the third embodiment can also provide the same effects as those of the first embodiment. For example, since the graphic representation representing the noise level included in the current waveform of the biological signal is displayed at the location corresponding to the peak of the current waveform, it is possible to intuitively ascertain the degree of influence of noise on the current waveform of the biological signal. Furthermore, in the third embodiment, by displaying the graphic representation representing the noise level of the past current waveform, it is possible to intuitively determine whether or not the noise included in the present current waveform is relatively large.

Note that, in the above-described embodiments, examples in which the graphic representation representing the noise level is displayed so as to be superimposed over the current waveform calculated based on the biomagnetism measured by the biometric information measurement device 100 have been described. However, the method of superimposing the graphic representation representing the noise level over the current waveform and displaying the superimposed image described in the above-described embodiments may also be applied to evaluation of a potential waveform of a biopotential of an electrocardiograph or the like.

In the above-described embodiments, examples in which a bioelectric current signal calculated from measured magnetism is processed as a biological signal have been described; however, a measured biomagnetic signal may be processed as a biological signal.

The present disclosure has, for example, the following aspects:

<1> A biometric information display device including:
circuitry; and
a memory storing computer-executable instructions that cause the circuitry to:
display at least one biological signal waveform including a biological signal measured; and
display a graphic representation representing a noise level included in the biological signal waveform at a location corresponding to a peak of the biological signal waveform.

<2> The biometric information display device according to above <1>, in which the graphic representation has a shape orthogonal to an axis representing time with respect to the biological signal waveform and having a length corresponding to a magnitude of the noise level.

<3> The biometric information display device according to above <1> or <2>, in which the circuitry is caused to:
calculate the noise level for a designated portion of time during a measurement period in which the biological signal is measured; and
display the graphic representation representing the noise level which has been calculated.

<4> The biometric information display device according to above <3>, in which the circuitry is caused to calculate, as the noise level, a root mean square of the biological signal, a difference between a maximum value and a minimum value of the biological signal, a maximum amplitude of the biological signal, a maximum value of a difference between peak values of the biological signal having different polarities and being adjacent to each other in an axis representing time, or a standard deviation of the biological signal, for the designated portion of time.

<5> The biometric information display device according to above <3> or <4>, in which the circuitry is caused to multiply the noise level which has been calculated by n (n is a value larger than 1) and display the noise level multiplied by n.

<6> The biometric information display device according to above <5>, in which the circuitry is caused to display the graphic representation with one end at the location corresponding to the peak and another end extending toward a baseline which is displayed parallel to an axis representing time, or the graphic representation with one end on the baseline and another end extending toward the peak.

<7> The biometric information display device according to any one of above <3> to <6>, in which the circuitry is caused to:
display the biological signal waveform that is a given one of a plurality of biological signal waveforms each including a corresponding one of a plurality of biological signals measured at different times;
calculate the noise level for each of the plurality of biological signals for the designated portion of time; and
display both the graphic representation representing the noise level of one of the biological signals in the designated portion of time used for generating the given one of the biological signal waveforms and the graphic representation representing the noise level of one of the biological signals in the designated portion of time used for generating a biological signal waveform other than the given one of the biological signal waveforms.

<8> The biometric information display device according to any one of above <3> to <7>, in which the designated portion of time is set to a time period during which the biological signal of interest does not appear in the measurement period.

<9> The biometric information display device according to any one of above <1> to <8>, in which the biological signal is a biomagnetic signal or a current signal calculated from biomagnetism measured.

<10> The biometric information display device according to any one of above <1> to <9>, in which the biological signal is a signal derived from skeletal muscle, a signal derived from cardiac muscle, a signal derived from smooth muscle, or a signal derived from a nerve of a living body.

<11> The biometric information display device according to any one of above <1> to <10>, in which the circuitry is caused to display, as the at least one biological signal waveform, a plurality of biological signal waveforms each including one of a plurality of biological signals measured at a plurality of measurement locations, and a color of the biological signal waveforms each having the noise level of equal to or higher than a preset threshold value is set different from a color of the biological signal waveforms each having the noise level of lower than the preset threshold value.

<12> A system including:
the biometric information display device of any one of above <1> to <11>; and
a biomagnetism measurement device including: circuitry; and
a memory storing computer-executable instructions that cause the circuitry to measure biomagnetism to generate the biological signal.

<13> A biometric information display method including:
displaying at least one biological signal waveform including a biological signal measured; and
displaying a graphic representation representing a noise level included in the biological signal waveform at a location corresponding to a peak of the biological signal waveform.

<14> A non-transitory computer-readable recording medium storing a program for displaying biometric information, the program, when executed by circuitry of a computer, causing the circuitry to execute:
a waveform display process of displaying at least one biological signal waveform including a biological signal measured; and
a noise level display process of displaying a graphic representation representing a noise level included in the biological signal waveform at a location corresponding to a peak of the biological signal waveform.

Although the present disclosure has been described above using the embodiments, the present disclosure is by no means limited to the requirements illustrated in the above embodiments. These points can be changed within the scope of the present disclosure, and can be appropriately determined according to the mode of implementation.

The present disclosure enables a user to intuitively ascertain a degree of influence of noise on a current waveform of a biological signal.

What is claimed is:

1. A biometric information display device comprising:
circuitry; and
a memory storing computer-executable instructions that cause the circuitry to:
display at least one biological signal waveform including a biological signal measured; and
display a graphic representation representing a noise level included in the biological signal waveform at a location corresponding to a peak of the biological signal waveform.

2. The biometric information display device according to claim 1, wherein the graphic representation has a shape orthogonal to an axis representing time with respect to the biological signal waveform and having a length corresponding to a magnitude of the noise level.

3. The biometric information display device according to claim 1, wherein the circuitry is caused to:
calculate the noise level for a designated portion of time during a measurement period in which the biological signal is measured; and
display the graphic representation representing the noise level which has been calculated.

4. The biometric information display device according to claim 3, wherein the circuitry is caused to calculate, as the noise level, a root mean square of the biological signal, a difference between a maximum value and a minimum value of the biological signal, a maximum amplitude of the biological signal, a maximum value of a difference between peak values of the biological signal having different polarities and being adjacent to each other in an axis representing time, or a standard deviation of the biological signal, for the designated portion of time.

5. The biometric information display device according to claim 3, wherein the circuitry is caused to multiply the noise level which has been calculated by n (n is a value larger than 1) and display the noise level multiplied by n.

6. The biometric information display device according to claim 5, wherein the circuitry is caused to display the graphic representation with one end at the location corresponding to the peak and another end extending toward a baseline which is displayed parallel to an axis representing time, or the graphic representation with one end on the baseline and another end extending toward the peak.

7. The biometric information display device according to claim 3, wherein the circuitry is caused to:
display the biological signal waveform that is a given one of a plurality of biological signal waveforms each including a corresponding one of a plurality of biological signals measured at different times;
calculate the noise level for each of the plurality of biological signals for the designated portion of time; and
display both the graphic representation representing the noise level of one of the biological signals in the designated portion of time used for generating the given one of the biological signal waveforms and the graphic representation representing the noise level of one of the biological signals in the designated portion of time used for generating a biological signal waveform other than the given one of the biological signal waveforms.

8. The biometric information display device according to claim 3, wherein the designated portion of time is set to a time period during which the biological signal of interest does not appear in the measurement period.

9. The biometric information display device according to claim 1, wherein the biological signal is a biomagnetic signal or a current signal calculated from biomagnetism measured.

10. The biometric information display device according to claim 1, wherein the biological signal is a signal derived from skeletal muscle, a signal derived from cardiac muscle, a signal derived from smooth muscle, or a signal derived from a nerve of a living body.

11. The biometric information display device according to claim 1, wherein the circuitry is caused to display, as the at least one biological signal waveform, a plurality of biological signal waveforms each including one of a plurality of biological signals measured at a plurality of measurement locations, and a color of the biological signal waveforms each having the noise level of equal to or higher than a preset threshold value is set different from a color of the biological signal waveforms each having the noise level of lower than the preset threshold value.

12. A system comprising:
the biometric information display device of claim 1; and
a biomagnetism measurement device including:
circuitry; and
a memory storing computer-executable instructions that cause the circuitry to measure biomagnetism to generate the biological signal.

13. A biometric information display method comprising:
displaying at least one biological signal waveform including a biological signal measured; and
displaying a graphic representation representing a noise level included in the biological signal waveform at a location corresponding to a peak of the biological signal waveform.

14. A non-transitory computer-readable recording medium storing a program for displaying biometric information, the program, when executed by circuitry of a computer, causing the circuitry to execute:
a waveform display process of displaying at least one biological signal waveform including a biological signal measured; and
a noise level display process of displaying a graphic representation representing a noise level included in the biological signal waveform at a location corresponding to a peak of the biological signal waveform.
